(19) United States
(12) Patent Application Publication
(10) Pub. No.: US 2025/0266141 A1
(43) Pub. Date: Aug. 21, 2025

(54) MEDICAL TREATMENT ASSIST APPARATUS, MEDICAL TREATMENT ASSIST METHOD, AND RECORDING MEDIUM

(71) Applicant: NEC Corporation, Tokyo (JP)

(72) Inventors: Hirokazu SAKAMOTO, Tokyo (JP); Tomoharu Kiyuna, Tokyo (JP); Tatsu Kimura, Tokyo (JP)

(73) Assignee: NEC Corporation, Tokyo (JP)

(21) Appl. No.: 19/050,252

(22) Filed: Feb. 11, 2025

(30) Foreign Application Priority Data
Feb. 21, 2024 (JP) 2024-024888

(51) Int. Cl. G16H 20/00; G16H 10/60; G16H 50/20; G16H 80/00

(52) U.S. Cl. CPC G16H 20/00; G16H 10/60; G16H 50/20; G16H 80/00

(57) ABSTRACT

This medical treatment assist apparatus includes: an acquiring section for acquiring medical information regarding a patient, content information which indicates the content of an exchange in a medical examination between the patient and a person examining the patient, and state information regarding the feelings of the patient in the medical examination; an explanatory text generating section for generating explanatory text regarding a matter which is inferred to make the patient feel uneasy, with use of a language model generated by machine learning, the medical information, the content information, and the state information; and an outputting section for outputting the explanatory text.

### CROSS REFERENCE TO RELATED APPLICATIONS

This application is based upon and claims the benefit of priority from Japanese Patent Application No. 2024-024888 filed on Feb. 21, 2024, the disclosure of which is incorporated herein in its entirety by reference.

### TECHNICAL FIELD

The present disclosure relates to a medical treatment assist apparatus, a medical treatment assist method, and a recording medium.

### BACKGROUND ART

Medical treatment assist techniques are known. Examples of medical treatment assist techniques include the technique disclosed in Patent Literature 1. Patent Literature 1 discloses an information processing apparatus for: receiving speech data or text data in telemedicine which is transmitted from a terminal on a doctor's end to a terminal on a patient's end; and in a case where the received data contains a medical term, outputting, to the terminal on the patient's end, glossary data for medical terms together with the speech data or the text data.

### CITATION LIST

### Patent Literature

### [Patent Literature 1]

Japanese Patent Application Publication Tokukai No. 2020-113003

### SUMMARY OF INVENTION

### Technical Problem

In medical treatment, in some cases, a patient feels uneasy because, for example, the patient cannot understand the content of an explanation made by a doctor or the like. There is a problem of being impossible, through the technique disclosed in Patent Literature 1, to alleviate an uneasy feeling that a patient experiences in medical treatment.

The present disclosure has been made in view of the above problem, and an example object thereof is to provide a technique for alleviating an uneasy feeling that a patient experiences in medical treatment.

### Solution to Problem

A medical treatment assist apparatus in accordance with an example aspect of the present disclosure includes at least one processor, and the at least one processor carries out: an acquiring process of acquiring medical information regarding a patient, content information which indicates content of an exchange in a medical examination between the patient and a person examining the patient, and state information regarding feelings of the patient in the medical examination; an explanatory text generating process of generating explanatory text regarding a matter which is inferred to make the patient feel uneasy, with use of a language model generated by machine learning, the medical information, the content information, and the state information; and an outputting process of outputting the explanatory text.

A medical treatment assist method in accordance with an example aspect of the present disclosure includes: at least one processor acquiring medical information regarding a patient, content information which indicates content of an exchange in a medical examination between the patient and a person examining the patient, and state information regarding feelings of the patient in the medical examination; the at least one processor generating explanatory text regarding a matter which is inferred to make the patient feel uneasy, with use of a language model generated by machine learning, the medical information, the content information, and the state information; and the at least one processor outputting the explanatory text.

A recording medium in accordance with an example aspect of the present disclosure is a recording medium having recorded thereon a medical treatment assist program for causing a computer to function as a medical treatment assist apparatus, and the medical treatment assist program causes the computer to carry out: an acquiring process of acquiring medical information regarding a patient, content information which indicates content of an exchange in a medical examination between the patient and a person examining the patient, and state information regarding feelings of the patient in the medical examination; an explanatory text generating process of generating explanatory text regarding a matter which is inferred to make the patient feel uneasy, with use of a language model generated by machine learning, the medical information, the content information, and the state information; and an outputting process of outputting the explanatory text.

### Advantageous Effects of Invention

An example aspect of the present disclosure provides an example advantage of making it possible to provide a technique for alleviating an uneasy feeling that a patient experiences in medical treatment.

### BRIEF DESCRIPTION OF DRAWINGS

FIG. 1 is a block diagram illustrating a configuration of a medical treatment assist apparatus in accordance with the present disclosure.

FIG. 2 is a flowchart illustrating a flow of a medical treatment assist method in accordance with the present disclosure.

FIG. 3 is a block diagram illustrating a configuration of an information processing apparatus in accordance with the present disclosure.

FIG. 4 is a diagram illustrating a functional configuration of a control section in accordance with the present disclosure.

FIG. 5 is a diagram illustrating a specific example of output information outputted by an output control section in accordance with the present disclosure.

FIG. 6 is a flowchart illustrating an example flow of the medical treatment assist method in accordance with the present disclosure.

FIG. 7 is a block diagram illustrating a configuration of a computer which functions as the medical treatment assist apparatus or the information processing apparatus in accordance with the present disclosure.

### EXAMPLE EMBODIMENTS

The following description will discuss example embodiments of the present invention. However, the present invention is not limited to the example embodiments described below, but can be altered by a skilled person in the art within the scope of the claims. For example, any embodiment derived by appropriately combining techniques (some or all of products or methods) adopted in differing example embodiments described below can be within the scope of the present invention. Further, any embodiment derived by appropriately omitting one or more of the techniques adopted in differing example embodiments described below can be within the scope of the present invention. Furthermore, the advantage mentioned in each of the example embodiments described below is an example advantage expected in that example embodiment, and does not define the extension of the present invention. That is, any embodiment which does not provide the example advantages mentioned in the example embodiments described below can also be within the scope of the present invention.

### First Example Embodiment

The following description will discuss a first example embodiment, which is an example embodiment of the present invention, in detail with reference to the drawings. The present example embodiment is basic to each of the example embodiments which will be described later. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### (Configuration of Medical Treatment Assist Apparatus)

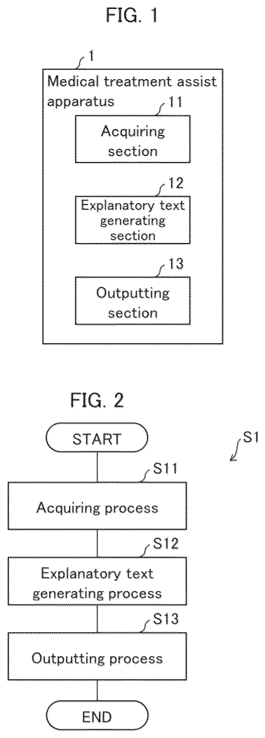

The configuration of a medical treatment assist apparatus 1 is described here with reference to FIG. 1. FIG. 1 is a block diagram illustrating the configuration of the medical treatment assist apparatus 1. The medical treatment assist apparatus 1 includes an acquiring section 11, an explanatory text generating section 12, and an outputting section 13, as illustrated in FIG. 1.

The acquiring section 11 acquires medical information regarding a patient, content information indicating the content of an exchange in a medical examination between the patient and a person examining the patient, and state information regarding the feelings of the patient in the medical examination. The explanatory text generating section 12 generates explanatory text regarding a matter which is inferred to make the patient feel uneasy, with use of a language model generated by machine learning, the medical information, the content information, and the state information. The outputting section 13 outputs the explanatory text generated by the explanatory text generating section 12.

### (Example Advantage of Medical Treatment Assist Apparatus)

As above, the medical treatment assist apparatus 1 includes: an acquiring section 11 for acquiring medical information regarding a patient, content information indicating the content of an exchange in a medical examination between the patient and a person examining the patient, and state information regarding the feelings of the patient in the medical examination; an explanatory text generating section 12 for generating explanatory text regarding a matter which is inferred to make the patient feel uneasy, with use of a language model generated by machine learning, the medical information, the content information, and the state information; and an outputting section 13 for outputting the explanatory text. Thus, the medical treatment assist apparatus 1 provides an example advantage of making it possible to alleviate an uneasy feeling that a patient experiences in medical treatment.

### (Flow of Medical Treatment Assist Method)

The flow of a medical treatment assist method S1 is described here with reference to FIG. 2. FIG. 2 is a flowchart illustrating the flow of the medical treatment assist method S1. The medical treatment assist method S1 includes an acquiring process S11, an explanatory text generating process S12, and an outputting process S13, as illustrated in FIG. 2.

In the acquiring process S11, at least one processor acquires medical information regarding a patient, content information indicating the content of an exchange in a medical examination between the patient and a person examining the patient, and state information regarding the feelings of the patient in the medical examination. In the explanatory text generating process S12, the at least one processor generates explanatory text regarding a matter which is inferred to make the patient feel uneasy, with use of a language model generated by machine learning, the medical information, the content information, and the state information. In the outputting process S13, the at least one processor outputs the explanatory text.

### (Example Advantage of Medical Examination Assist Method)

As above, the medical treatment assist method S1 includes: an acquiring process S11 of at least one processor acquiring medical information regarding a patient, content information indicating the content of an exchange in a medical examination between the patient and a person examining the patient, and state information regarding the feelings of the patient in the medical examination; an explanatory text generating process S12 of the at least one processor generating explanatory text regarding a matter which is inferred to make the patient feel uneasy, with use of a language model generated by machine learning, the medical information, the content information, and the state information; and an outputting process S13 of the at least one processor outputting the explanatory text. Thus, the medical treatment assist method S1 provides an example advantage of making it possible to alleviate an uneasy feeling that a patient experiences in medical treatment.

### Second Example Embodiment

The following description will discuss a second example embodiment, which is an example embodiment of the present invention, in detail with reference to the drawings. A component having the same function as a component described in the above example embodiment is assigned the same reference sign, and the description thereof is omitted where appropriate. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### (Configuration of Information Processing Apparatus)

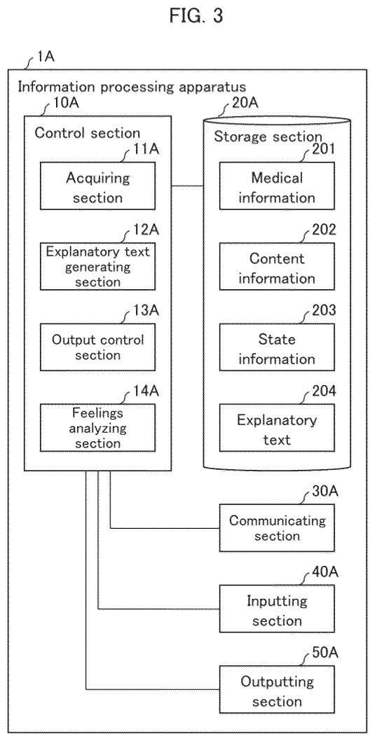

The configuration of an information processing apparatus 1A is described here with reference to FIG. 3. FIG. 3 is a block diagram illustrating the configuration of the information processing apparatus 1A. The information processing apparatus 1A is an example medical treatment assist apparatus in accordance with the present disclosure. The information processing apparatus 1A includes a control section 10A, a storage section 20A, a communicating section 30A, an inputting section 40A, and an outputting section 50A.

### (Communicating Section)

The communicating section 30A communicates with an apparatus external to the information processing apparatus 1A over a communication line. A specific configuration of the communication line does not limit the present example embodiment, but examples of the communication line include a wireless local area network (LAN), a wired LAN, a wide area network (WAN), a public network, a mobile data communication network, and a combination thereof. The communicating section 30A transmits, to another apparatus, data supplied from the control section 10A, and supplies the control section 10A with data received from another apparatus.

### (Inputting Section)

The inputting section 40A is a component for accepting an input to the information processing apparatus 1A, and includes inputting equipment such as, for example, a keyboard, a mouse, a touch panel, a camera, or a microphone. Further, the inputting section 40A may be a component for accepting data from inputting equipment via an interface such as, for example, a universal serial bus (USB).

### (Outputting Section)

The outputting section 50A is a component for producing an output from the information processing apparatus 1A, and includes outputting equipment such as, for example, a display, a printer, a touch panel, or a speaker. The outputting section 50A may be a component which, for example, includes an interface such as a USB and outputs data to outputting equipment via the interface.

### (Storage Section)

In the storage section 20A, various kinds of information to be referred to by the control section 10A are stored. Examples of such information include medical information 201, content information 202, state information 203, and explanatory text 204.

### (Medical Information)

The medical information 201 is information regarding medical care for a patient. Examples of the medical information 201 include medical record information. As an example, the medical information 201 includes at least one selected from the group consisting of personal information regarding the patient, information on findings shown by a medical examination performed on the patient, and medical history information regarding the patient. The personal information regarding the patient is personal information regarding the patient, and includes, for example, information indicating the age, the gender, the amount of smoking, etc. of the patient. The information on findings shown by a medical examination performed on the patient is information indicating findings made by a medical service worker, such as a doctor, and includes, for example, information obtained by the medical service worker through a medical interview or an inspection. The information obtained through a medical interview or an inspection may be, for example, text “pressure on the abdomen causes a pain”. The medical history information regarding the patient is information regarding the history of disease of the patient, and includes, for example, information indicating the past medical history of the patient, the past medical history of the family, the amount of smoking of the patient, etc.

The medical information 201 may include a diagnosis target image of a patient. As an example, the diagnosis target image includes at least one of images which are an X-ray image, an endoscope image, a pathological image, an MRI image, and a CT image. In the storage section 20A, respective pieces of medical information 201 regarding a plurality of patients are stored.

### (Content Information)

The content information 202 is information indicating the content of an exchange in a medical examination between a patient and a person examining the patient in a medical examination. The person examining the patient is, for example, a medical service worker, such as a doctor. The content information 202 may be, for example, speech data obtained by picking up a conversation in a medical examination between the patient and the medical service worker. In this case, the conversation between the patient and the medical service worker includes, for example, a complaint and a question made by the patient. Further, the content information 202 may be, for example, information indicating the content of a complaint and a question that the patient writes in an interview sheet.

The content information 202 may be data in text format, or may be speech data. The content information 202 is, for example, text which includes a sentence expressing a question such as “Is it safe? What surgery or test should I undergo?”.

### (State Information)

The state information 203 is information regarding the feelings of a patient. As an example, the state information 203 includes information which indicates at least one selected from the group consisting of the facial expression, the manner of speaking, a vital sign, and a feelings analysis result of the patient in a medical examination. As an example, the feelings analysis is carried out by a feelings analyzing section 14A, which will be described later. In other words, the state information 203 may include information indicating the result of an analysis, carried out by the feelings analyzing section 14A, of the feelings of the patient. As an example, the information indicating the result of an analysis of the feelings indicates a state of “tenseness” or “uneasiness”.

### (Explanatory Text)

The explanatory text 204 is text regarding a matter which is inferred to make a patient feel uneasy. As an example, the explanatory text 204 is text in which the content of therapy is plainly explained or text for alleviating the uneasy feeling of the patient. More specifically, the explanatory text 204 includes, for example, a sentence such as “Acute appendicitis is suspected. The therapy of this disease has a very high success rate.” and a sentence such as “In a case of treatment by surgery, the surgery is such that under anesthesia, a small incision is made in the abdomen to remove a lesion via a camera and an instrument. The surgical scar is small, and the hospital stay is short and the recovery is quick. First of all, a CT examination will be made in order to identify the symptom.”.

### (Control Section)

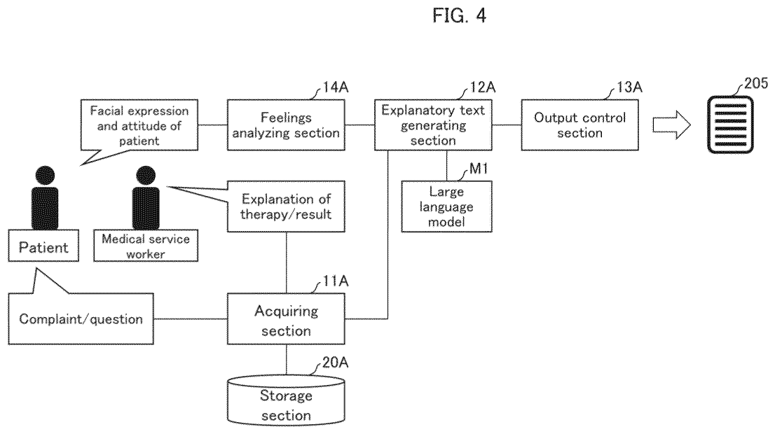

FIG. 4 is a diagram illustrating a functional configuration of the control section 10A. The control section 10A includes an acquiring section 11A, an explanatory text generating section 12A, an output control section 13A, and a feelings analyzing section 14A.

### (Acquiring Section)

The acquiring section 11A acquires the medical information 201 regarding a patient being examined and the content information 202 indicating the content of an exchange in a medical examination between the patient and a medical service worker or the like, and provides the explanatory text generating section 12A with the medical information 201 and the content information 202 acquired. As an example, the acquiring section 11A may acquire the medical information 201 and the content information 202 by retrieving the medical information 201 and the content information 202 from a storage location (which may be storage in the information processing apparatus 1A, or may be storage external to the information processing apparatus 1A) designated by a user of the information processing apparatus 1A. The acquiring section 11A may acquire the medical information 201 and the content information 202 by receiving the medical information 201 and the content information 202 from another apparatus via the communicating section 30A. The acquiring section 11A may acquire the medical information 201 and the content information 202 inputted to the inputting section 40A.

As an example, the acquiring section 11A acquires speech data indicating a speech picked up in a medical examination, and converts the speech data into the content information 202 in text format. In this case, the acquiring section may 11A carry out speaker recognition based on the speech data and transcribe, speaker by speaker, what speakers say, to generate the content information 202 which represents the content of a speaker-by-speaker speech.

### (Feelings Analyzing Section)

The feelings analyzing section 14A analyzes the feelings of a patient in a medical examination, and generates the result of the analysis. As an example, the feelings analyzing section 14A may analyze at least one selected from the group consisting of speech data regarding the patient picked up in the medical examination and image data captured in the medical examination, to analyze the feelings of the patient. The image data may be data representing a still image, or may be data representing a moving image. Examples of an approach for the feelings analysis may include an approach of using dictionaries prepared in advance and an approach of using a trained model generated by machine learning. In a case of using the trained model, the input to the trained model includes, for example, at least one selected from the group consisting of text, speech data, and image data. The output t from the trained model includes information indicating the result of classification of the feelings.

The feelings analyzing section 14A provides the explanatory text generating section 12A with the analysis result. The feelings analyzing section 14A may output the analysis result by writing the analysis result in a storage location (which may be storage in the information processing apparatus 1A, or may be storage external to the information processing apparatus 1A) designated by a user of the information processing apparatus 1A. Further, the feelings analyzing section 14A may transmit the analysis result to another apparatus via the communicating section 30A, or may output the analysis result to outputting equipment such as a display.

### (Explanatory Text Generating Section)

The explanatory text generating section 12A acquires the state information 203. As an example, the explanatory text generating section 12A may acquire the state information 203 by retrieving the state information 203 from a storage location (which may be storage in the information processing apparatus 1A, or may be storage external to the information processing apparatus 1A) designated by a user of the information processing apparatus 1A. The explanatory text generating section 12A may acquire the state information 203 by receiving the state information 203 from another apparatus via the communicating section 30A. The explanatory text generating section 12A may acquire the state information 203 inputted to the inputting section 40A.

As an example, the state information 203 includes the analysis result provided by the feelings analyzing section 14A. Further, the state information 203 may include information inputted by a medical service worker who examines a patient with use of the inputting section 40A, or information transmitted to the information processing apparatus 1A from a communication apparatus operated by the medical service worker. In this case, the medical service worker examining a patient observes the facial expression, etc. of the patient in the medical examination, and in a case where, for example, it is considered that the patient is feeling tense or uneasy and/or the patient does not understand the explanation, the medical service worker inputs information to that effect.

The state information 203 may include information inputted by the patient with use of the inputting section 40A, or information transmitted to the information processing apparatus 1A from a communication apparatus operated by the patient. In this case, for example, if the patient cannot t understand the explanation made by the medical service worker and/or the patient feels tense or uneasy about therapy, the patient inputs information to that effect.

The explanatory text generating section 12A generates the explanatory text 204 with use of a large language model M1 generated by machine learning, the medical information 201, the content information 202, and the state information 203. The large language model M1 is an example of the language model in accordance with the present disclosure. The large language model M1 may be stored in the storage section 20A of the information processing apparatus 1A, or may be stored in an apparatus other than the information processing apparatus 1A. The large language model M1 being stored in the storage section 20A means that parameters defining the large language model M1 are stored in the storage section 20A.

The large language model M1 is a language model which is formed by an artificial neural network having a great number of parameters and which is generated by machine learning. Examples of the large language model M1 includes, but is not limited to, generative AI such as Chat Generative Pre-Trained Transformer (ChatGPT) or Generative Pre-trained Transformer 4 (GPT-4), and such generative AI having been fine-tuned with data related to medical care.

Input information inputted by the explanatory text generating section 12A to the large language model M1 includes at least one selected from the group consisting of the medical information 201, the content information 202, and the state information 203. The input information may include information indicating explanatory text generation instructions. For example, the information may be text such as “Please make plain the explanation of the therapy” or “please generate explanatory text for alleviating the uneasy feeling of the patient”. Output information outputted from the large language model M1 includes the explanatory text 204.

As an example, in a case where the large language model M1 is stored in an apparatus other than the information processing apparatus 1A, the explanatory text generating section 12A transmits the input information via the communicating section 30A to the apparatus in which the large language model M1 is stored, to input the input information to the large language model M1. In this case, the explanatory text generating section 12A receives information outputted by the large language model M1 from that apparatus via the communicating section 30A.

The explanatory text generating section 12A may refer to the state information 203 to determine whether the patient is feeling uneasy. In this case, the explanatory text generating section 12A may refer to the state information 203 to determine whether the patient is feeling uneasy, and generate the explanatory text 204 in a case of a determination that the patient is feeling uneasy.

### (Output Control Section)

The output control section 13A outputs the output information 205 which includes the explanatory text 204 generated by the explanatory text generating section 12A. As an example, the output control section 13A may output the output information 205 by writing the output information 205 in a storage location (which may be storage in the information processing apparatus 1A, or may be storage external to the information processing apparatus 1A) designated by a user of the information processing apparatus 1A. Further, the output control section 13A may transmit the output information 205 via the communicating section 30A, or may output the output information 205 to outputting equipment such as a display.

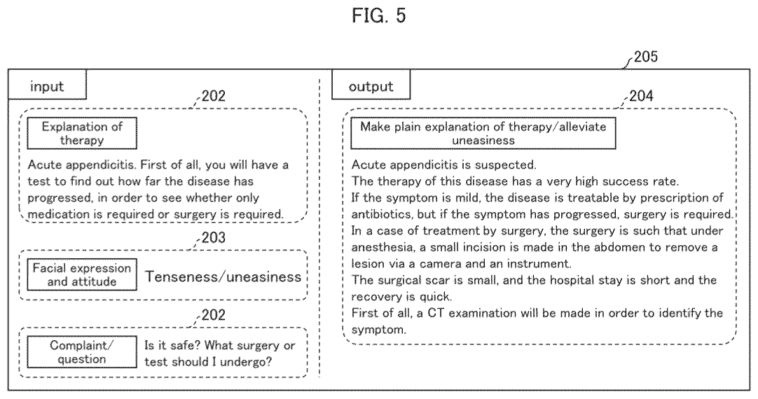

FIG. 5 is a diagram illustrating a specific example of the output information 205 outputted by the output control section 13A. As an example, the output control section 13A may display the output information 205 illustrated in FIG. 5 on displaying equipment. In this case, a screen displayed on the displaying equipment includes the explanatory text 204. The screen further includes the content information 202 and the state information 203, which are inputs to the information processing apparatus 1A. As illustrated in FIG. 5, the output control section 13A may output at least one selected from the group consisting of the medical information 201, the content information 202, and the state information 203, together with the explanatory text 204.

In the example of FIG. 5, the output information 205 includes the content information 202 in which the “explanation of therapy” is such that “Acute appendicitis. First of all, you will have a test to find out how far the disease has progressed, in order to see whether only medication is required or surgery is required.” Further, the output information 205 includes the state information 203 in which the “facial expression and attitude” is “tenseness/uneasiness”. Further, the output information 205 includes the content information 202 in which the “complaint/question” is such that “Is it safe? What surgery or test should I undergo?”.

Furthermore, in the example of FIG. 5, the output information 205 includes the explanatory text 204 which is such that “Acute appendicitis is suspected. The therapy of this disease has a very high success rate. If the symptom is mild, the disease is treatable by prescription of antibiotics, but if the symptom has progressed, surgery is required. In a case of treatment by surgery, the surgery is such that under anesthesia, a small incision is made in the abdomen to remove a lesion via a camera and an instrument. The surgical scar is small, and the hospital stay is short and the recovery is quick. First of all, a CT examination will be made in order to identify the symptom.”.

For example, the explanatory text 204 is used in decision-making by a patient regarding medical treatment. As an example, it is possible for a patient to check the explanatory text 204 to decide an action such as consulting a doctor again about the therapeutic strategy.

### (Flow of Medical Treatment Assist Method)

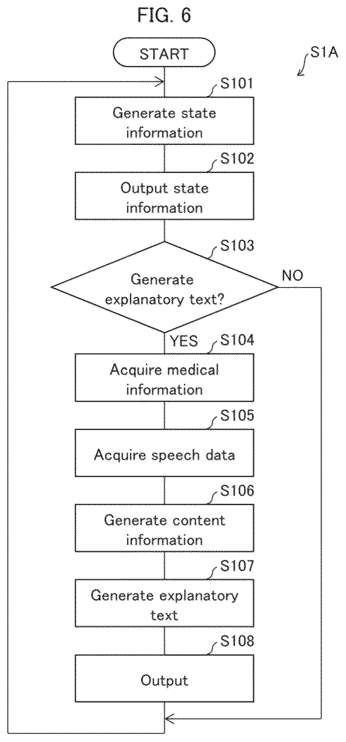

FIG. 6 is a flowchart illustrating an example flow of a medical treatment assist method S1A carried out by the information processing apparatus 1A. The steps included in the flowchart of FIG. 6 may be carried out in parallel with each other or in a different order. In step S101, the feelings analyzing section 14A analyzes the facial expression, the content of a speech, and the like of a patient in a medical examination, to generate an analysis result, which is the state information 203.

In step S102, the feelings analyzing section 14A outputs the state information 203, which is the analysis result, to outputting equipment. As an example, the feelings analyzing section 14A may output the state information 203 to a display, and display the state information 203 on the display.

A medical service worker examining the patient checks the state information 203 displayed on the display, to determine whether to output the explanatory text 204. In a case of outputting the explanatory text 204, the medical service worker operates an operation element or the like which is connected to the inputting section 40A, to input, to the information processing apparatus 1A, an instruction to output the explanatory text 204. For example, in a case where the state information 203 outputted includes at least one selected from the group consisting of information indicating “tenseness” and information indicating “uneasiness”, the medical service worker determines that the explanatory text 204 should be outputted. In a case of a determination that the explanatory text 204 does not need to be outputted, the medical service worker does not input the output instruction. For example, in a case where the state information 203 outputted does not include information indicating “tenseness” or information indicating “uneasiness”, the medical service worker determines that the explanatory text 204 should not be outputted.

In step S103, the explanatory text generating section 12A determines whether to generate the explanatory text 204. As an example, the explanatory text generating section 12A makes this determination according to whether the output instruction is inputted by a user such as the medical service worker or the like. In a case of a determination that the explanatory text 204 should be generated, the explanatory text generating section 12A moves to the process of step S104. In a case of a determination that the explanatory text 204 should not be generated, the explanatory text generating section 12A skips the processes of step S104 to step S108 and returns to the process of step S101.

In step S103, the determination approach carried out by the explanatory text generating section 12A is not limited to the above-described approach, but the explanatory text generating section 12A may use another approach to determine whether to generate the explanatory text 204. As an example, the explanatory text generating section 12A may refer to the state information 203 generated by the feelings analyzing section 14A, to determine whether to output the explanatory text 204. In this case, as an example, in a case where the state information 203 includes at least one selected from the group consisting of information indicating “tenseness” and information indicating “uneasiness”, the explanatory text generating section 12A may determine that the explanatory text 204 should be outputted. Further, as an example, in a case where the state information 203 does not include information indicating “tenseness” or information indicating “uneasiness”, the explanatory text generating section 12A may determine that the explanatory text 204 should not be outputted.

In step S104, the acquiring section 11A acquires the medical information 201 from the storage section 20A. In step S105, the acquiring section 11A acquires speech data picked up in a medical examination. The speech data is data representing a conversation in the medical examination between the patient and the medical service worker. In step S106, the acquiring section 11A performs speaker recognition processing on the speech data and transcribes, speaker by speaker, what the speakers say, to generate the content information which indicates the content of a speaker-by-speaker speech. The acquiring section 11A provides the explanatory text generating section 12A with the medical information 201 and the content information 202 generated.

In step S107, the explanatory text generating section 12A inputs the medical information 201, the content information 202, and the state information 203 to the large language model M1, to generate the explanatory text 204. In step S108, the output control section 13A outputs the explanatory text 204. Upon the completion of the process of step S108, the control section 10A returns to the process of step S101.

### (Example Advantage of Information Processing Apparatus)

As above, the information processing apparatus 1A includes a feelings analyzing section 14A for analyzing the feelings of a patient in a medical examination, and the explanatory text generating section 12A acquires the state information 203 which includes an analysis result provided by the feelings analyzing section 14A. Thus, the information processing apparatus 1A provides an example advantage of making it possible to output the explanatory text 204 having reflected therein the result of analyzing the feelings of a patient and making it possible to alleviate tenseness or uneasiness of the patient by the patient checking the explanatory text 204 outputted.

In the information processing apparatus 1A, the acquiring section 11A acquires speech data representing a speech picked up in a medical examination, and the speech data acquired is converted into the content information 202 in text format. Thus, the information processing apparatus 1A provides an example advantage of making it possible to output the explanatory text 204 having reflected therein the content of a conversation made in the medical examination.

In the information processing apparatus 1A, speaker recognition is performed based on the speech data and what speakers say are transcribed speaker by speaker, so that the content information 202 indicating the content of a speaker-by-speaker speech is generated. Thus, the information processing apparatus 1A provides an example advantage of making it possible to output the explanatory text 204 having reflected therein the content of a speaker-by-speaker speech made in a medical examination.

In the information processing apparatus 1A, the explanatory text generating section 12A refers to the state information 203 to determine whether a patient is feeling uneasy, and generates the explanatory text 204 in a case of a determination that the patient is feeling uneasy. Thus, with the information processing apparatus 1A, it is possible to prevent unnecessary outputting of the explanatory text 204.

In the information processing apparatus 1A, the state information 203 includes at least one selected from the group consisting of the facial expression, the manner of speaking, a vital sign, and a feelings analysis result of a patient in a medical examination. Thus, the information processing apparatus 1A provides an example advantage of making it possible to output the explanatory text 204 having reflected therein information which includes at least one selected from the group consisting of the facial expression, the manner of speaking, a vital sign, and a feelings analysis result of the patient in the medical examination.

In the information processing apparatus 1A, the medical information 201 includes at least one selected from the group consisting of personal information regarding a patient, information on findings shown by a medical examination performed on the patient, and medical history information regarding the patient. Thus, the information processing apparatus 1A provides an example advantage of making it possible to output the explanatory text 204 having reflected therein at least on selected from the group consisting of the personal information regarding the patient, the information on findings shown by a medical examination performed on the patient, and medical history information regarding the patient.

In the information processing apparatus 1A, the explanatory text 204 is used in decision-making by a patient regarding a medical treatment. Thus, the information processing apparatus 1A enables the patient who undergoes medical treatment to more properly carry out decision-making.

### [Software Implementation Example]

Some or all of the functions of the medical treatment assist apparatus 1 and the information processing apparatus 1A (hereinafter, also referred to as “each apparatus above”) may be implemented by hardware such as an integrated circuit (IC chip), or may be implemented by software.

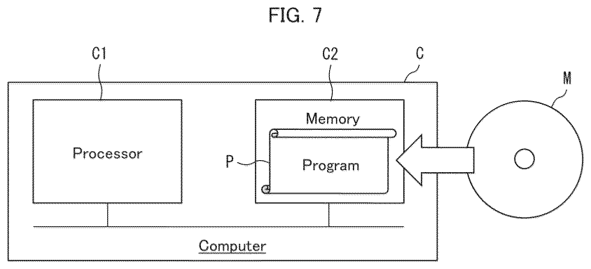

In the latter case, the each apparatus above is provided by, for example, a computer that executes instructions of a program that is software implementing the functions. An example (hereinafter, computer C) of such a computer is illustrated in FIG. 7. FIG. 7 is a block diagram illustrating a hardware configuration of the computer C which functions as each apparatus above.

The computer C includes at least one processor C1 and at least one memory C2. The memory C2 has recorded thereon a program P for causing the computer C to operate as each apparatus above. The processor C1 of the computer C retrieves the program P from the memory C2 and executes the program P, so that the functions of each apparatus above are implemented.

Examples of the processor C1 can include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 can include a flash memory, a hard disk drive (HDD), a solid state drive (SSD), and a combination thereof.

The computer C may further include a random access memory (RAM) into which the program P is loaded at the time of execution and in which various kinds of data are temporarily stored. The computer C may further include a communication interface via which data is transmitted to and received from another apparatus. The computer C may further include an input-output interface via which inputting-outputting equipment such as a keyboard, a mouse, a display, or a printer is connected.

The program P can be recorded on a non-transitory tangible recording medium M capable of being read by the computer C. Examples of such a recording medium M can include a tape, a disk, a card, a semiconductor memory, and a programmable logic circuit. The computer C can obtain the program P via such a recording medium M. The program P can be transmitted via a transmission medium. Examples of such a transmission medium can include a communication network and a broadcast wave. The computer C can obtain the program P also via such a transmission medium.

The above-described functions of each apparatus above may be implemented by a single processor provided in a single computer, may be implemented by the cooperation among a plurality of processors provided in a single computer, or may be implemented by the cooperation among a plurality of processors provided in a plurality of respective computers. Further, the program for causing each apparatus above to implement the above-described functions may be stored in a single memory provided in a single computer, may be stored in a distributed manner in a plurality of memories provided in a single computer, or may be stored in a distributed manner in a plurality of memories provided in a plurality of respective computers.

### Additional Remark A

The whole or part of the example embodiments disclosed above can be described as, but not limited to, the following supplementary notes.

### Supplementary Note A1

A medical treatment assist apparatus, including:
an acquiring means for acquiring medical information regarding a patient, content information which indicates content of an exchange in a medical examination between the patient and a person examining the patient, and state information regarding feelings of the patient in the medical examination;
an explanatory text generating means for generating explanatory text regarding a matter which is inferred to make the patient feel uneasy, with use of a language model generated by machine learning, the medical information, the content information, and the state information; and
an outputting means for outputting the explanatory text.

### Supplementary Note A2

The medical treatment assist apparatus described in supplementary note A1, further including
a feelings analyzing means for analyzing the feelings of the patient in the medical examination,
the acquiring means being configured to acquire the state information which includes an analysis result provided by the feelings analyzing means.

### Supplementary Note A3

The medical treatment assist apparatus described in supplementary note A1 or A2, in which
the acquiring means is configured to acquire speech data representing a speech picked up in the medical examination, and convert the speech data into the content information in text format.

### Supplementary Note A4

The medical treatment assist apparatus described in supplementary note A3, in which
the acquiring means is configured to carry out speaker recognition based on the speech data and transcribe, speaker by speaker, what speakers say, to generate the content information which represents content of a speaker-by-speaker speech.

### Supplementary Note A5

The medical treatment assist apparatus described in any one of supplementary notes A1 to A4, in which
the explanatory text generating means is configured to refer to the state information to determine whether the patient is feeling uneasy, and generate the explanatory text in a case of a determination that the patient is feeling uneasy.

### Supplementary Note A6

The medical treatment assist apparatus described in any one of supplementary notes A1 to A5, in which
the state information includes information which indicates at least one selected from the group consisting of a facial expression, a manner of speaking, a vital sign, and a feelings analysis result of the patient in the medical examination.

### Supplementary Note A7

The medical treatment assist apparatus described in any one of supplementary notes A1 to A6, in which
the medical information includes at least one selected from the group consisting of personal information regarding the patient, information on findings shown by a medical examination performed on the patient, and medical history information regarding the patient.

### Supplementary Note A8

The medical treatment assist apparatus described in any one of supplementary notes A1 to A7, in which
the explanatory text is used in decision-making by the patient regarding medical treatment.

### Additional Remark B

The whole or part of the example embodiments disclosed above can be described as, but not limited to, the following supplementary notes.

### Supplementary Note B1

A medical treatment assist method, including:
at least one processor acquiring medical information regarding a patient, content information which indicates content of an exchange in a medical examination between the patient and a person examining the patient, and state information regarding feelings of the patient in the medical examination;
the at least one processor generating explanatory text regarding a matter which is inferred to make the patient feel uneasy, with use of a language model generated by machine learning, the medical information, the content information, and the state information; and
the at least one processor outputting the explanatory text.

### Supplementary Note B2

The medical treatment assist method described in supplementary note B1, further including
the at least one processor analyzing the feelings of the patient in the medical examination,
in the acquiring, the at least one processor acquiring the state information which includes an analysis result provided by the feelings analyzing process.

### Supplementary Note B3

The medical treatment assist method described in supplementary note B1 or B2, in which
in the acquiring, the at least one processor acquires speech data representing a speech picked up in the medical examination, and converts the speech data into the content information in text format.

### Supplementary Note B4

The medical treatment assist method described in supplementary note B3, in which
in the acquiring, the at least one processor carries out speaker recognition based on the speech data and transcribes, speaker by speaker, what speakers say, to generate the content information which represents content of a speaker-by-speaker speech.

### Supplementary Note B5

The medical treatment assist method described in any one of supplementary notes B1 to B4, in which
in the generating, the at least one processor refers to the state information to determine whether the patient is feeling uneasy, and generates the explanatory text in a case of a determination that the patient is feeling uneasy.

### Supplementary Note B6

The medical treatment assist method described in any one of supplementary notes B1 to B5, in which
the state information includes information which indicates at least one selected from the group consisting of a facial expression, a manner of speaking, a vital sign, and a feelings analysis result of the patient in the medical examination.

### Supplementary Note B7

The medical treatment assist method described in any one of supplementary notes B1 to B6, in which
the medical information includes at least one selected from the group consisting of personal information regarding the patient, information on findings shown by a medical examination performed on the patient, and medical history information regarding the patient.

### Supplementary Note B8

The medical treatment assist method described in any one of supplementary notes B1 to B7, in which
the explanatory text is used in decision-making by the patient regarding medical treatment.

### Additional Remark C

The whole or part of the example embodiments disclosed above can be described as, but not limited to, the following supplementary notes.

### Supplementary Note C1

A medical treatment assist program for causing a computer to function as a medical treatment assist apparatus,
the medical treatment assist program causing the computer to function as:
an acquiring means for acquiring medical information regarding a patient, content information which indicates content of an exchange in a medical examination between the patient and a person examining the patient, and state information regarding feelings of the patient in the medical examination;
an explanatory text generating means for generating explanatory text regarding a matter which is inferred to make the patient feel uneasy, with use of a language model generated by machine learning, the medical information, the content information, and the state information; and
an outputting means for outputting the explanatory text.

### Supplementary Note C2

The medical treatment assist program described in supplementary note C1,
further causing the computer to function as
a feelings analyzing means for analyzing the feelings of the patient in the medical examination,
the acquiring means being configured to acquire the state information which includes an analysis result provided by the feelings analyzing means.

### Supplementary Note C3

The medical treatment assist program described in supplementary note C1 or C2, in which
the acquiring means is configured to acquire speech data representing a speech picked up in the medical examination, and convert the speech data into the content information in text format.

### Supplementary Note C4

The medical treatment assist program described in supplementary note C3, in which
the acquiring means is configured to carry out speaker recognition based on the speech data and transcribe, speaker by speaker, what speakers say, to generate the content information which represents content of a speaker-by-speaker speech.

### Supplementary Note C5

The medical treatment assist program described in any one of supplementary notes C1 to C4, in which
the explanatory text generating means is configured to refer to the state information to determine whether the patient is feeling uneasy, and generate the explanatory text in a case of a determination that the patient is feeling uneasy.

### Supplementary Note C6

The medical treatment assist program described in any one of supplementary notes C1 to C5, in which
the state information includes information which indicates at least one selected from the group consisting of a facial expression, a manner of speaking, a vital sign, and a feelings analysis result of the patient in the medical examination.

### Supplementary Note C7

The medical treatment assist program described in any one of supplementary notes C1 to C6, in which
the medical information includes at least one selected from group the consisting of personal information regarding the patient, information on findings shown by a medical examination performed on the patient, and medical history information regarding the patient.

### Supplementary Note C8

The medical treatment assist program described in any one of supplementary notes C1 to C7, in which
the explanatory text is used in decision-making by the patient regarding medical treatment.

### Additional Remark D

The whole or part of the example embodiments disclosed above can be described as, but not limited to, the following supplementary notes.

### Supplementary Note D1

A medical treatment assist apparatus, including
at least one processor, the at least one processor carrying out:
an acquiring process of acquiring medical information regarding a patient, content information which indicates content of an exchange in a medical examination between the patient and a person examining the patient, and state information regarding feelings of the patient in the medical examination;
an explanatory text generating process of generating explanatory text regarding a matter which is inferred to make the patient feel uneasy, with use of a language model generated by machine learning, the medical information, the content information, and the state information; and
an outputting process of outputting the explanatory text.

The medical treatment assist apparatus may further include a memory. The memory may have stored therein a program for causing the at least one processor to carry out each of the processes.

### Supplementary Note D2

The medical treatment assist apparatus described in supplementary note D1, in which
the at least one processor further carries out
a feelings analyzing process of analyzing the feelings of the patient in the medical examination, and
in the acquiring process, the at least one processor acquires the state information which includes an analysis result provided by the feelings analyzing process.

### Supplementary Note D3

The medical treatment assist apparatus described in supplementary note D1 or D2, in which
in the acquiring process, the at least one processor acquires speech data representing a speech picked up in the medical examination, and converts the speech data into the content information in text format.

### Supplementary Note D4

The medical treatment assist apparatus described in supplementary note D3, in which
in the acquiring process, the at least one processor carries out speaker recognition based on the speech data and transcribes, speaker by speaker, what speakers say, to generate the content information which represents content of a speaker-by-speaker speech.

### Supplementary Note D5

The medical treatment assist apparatus described in any one of supplementary notes D1 to D4, in which
in the explanatory text generating process, the at least one processor refers to the state information to determine whether the patient is feeling uneasy, and generates the explanatory text in a case of a determination that the patient is feeling uneasy.

### Supplementary Note D6

The medical treatment assist apparatus described in any one of supplementary notes D1 to D5, in which
the state information includes information which indicates at least one selected from the group consisting of a facial expression, a manner of speaking, a vital sign, and a feelings analysis result of the patient in the medical examination.

### Supplementary Note D7

The medical treatment assist apparatus described in any one of supplementary notes D1 to D6, in which
the medical information includes at least one selected from group the consisting of personal information regarding the patient, information on findings shown by a medical examination performed on the patient, and medical history information regarding the patient.

### Supplementary Note D8

The medical treatment assist apparatus described in any one of supplementary notes D1 to D7, in which
the explanatory text is used in decision-making by the patient regarding medical treatment.

### Additional Remark E

The whole or part of the example embodiments disclosed above can be described as, but not limited to, the following supplementary notes.

### Supplementary Note E1

A computer-readable non-transitory recording medium having recorded thereon a medical treatment assist program for causing a computer to function as a medical treatment assist apparatus,
the medical treatment assist program causing the computer to carry out:
an acquiring process of acquiring medical information regarding a patient, content information which indicates content of an exchange in a medical examination between the patient and a person examining the patient, and state information regarding feelings of the patient in the medical examination;
an explanatory text generating process of generating explanatory text regarding a matter which is inferred to make the patient feel uneasy, with use of a language model generated by machine learning, the medical information, the content information, and the state information; and
an outputting process of outputting the explanatory text.

### REFERENCE SIGNS LIST

1: Medical treatment assist apparatus
1A: Information processing apparatus
11, 11A: Acquiring section
12, 12A: Explanatory text generating section
13, 50A: Outputting section
13A: Output control section
14A: Feelings analyzing section

1. A medical treatment assist apparatus, comprising
at least one processor, the at least one processor carrying out:
an acquiring process of acquiring medical information regarding a patient, content information which indicates content of an exchange in a medical examination between the patient and a person examining the patient, and state information regarding feelings of the patient in the medical examination;
an explanatory text generating process of generating explanatory text regarding a matter which is inferred to make the patient feel uneasy, with use of a language model generated by machine learning, the medical information, the content information, and the state information; and
an outputting process of outputting the explanatory text.

2. The medical treatment assist apparatus according to claim 1, wherein
the at least one processor further carries out a feelings analyzing process of analyzing the feelings of the patient in the medical examination, and
in the acquiring process, the at least one processor acquires the state information which includes an analysis result provided by the feelings analyzing process.

3. The medical treatment assist apparatus according to claim 1, wherein
in the acquiring process, the at least one processor acquires speech data representing a speech picked up in the medical examination, and converts the speech data into the content information in text format.

4. The medical treatment assist apparatus according to claim 3, wherein
in the acquiring process, the at least one processor carries out speaker recognition based on the speech data and transcribes, speaker by speaker, what speakers say, to generate the content information which represents content of a speaker-by-speaker speech.

5. The medical treatment assist apparatus according to claim 1, wherein
in the explanatory text generating process, the at least one processor refers to the state information to determine whether the patient is feeling uneasy, and generates the explanatory text in a case of a determination that the patient is feeling uneasy.

6. The medical treatment assist apparatus according to claim 1, wherein
the state information includes information which indicates at least one selected from the group consisting of a facial expression, a manner of speaking, a vital sign, and a feelings analysis result of the patient in the medical examination.

7. The medical treatment assist apparatus according to claim 1, wherein
the medical information includes at least one selected from the group consisting of personal information regarding the patient, information on findings shown by a medical examination performed on the patient, and medical history information regarding the patient.

8. The medical treatment assist apparatus according to claim 1, wherein
the explanatory text is used in decision-making by the patient regarding medical treatment.

9. A medical treatment assist method, comprising:
at least one processor acquiring medical information regarding a patient, information which indicates content of an exchange in a medical examination between the patient and a person examining the patient, and state information regarding feelings of the patient in the medical examination;
the at least one processor generating explanatory text regarding a matter which is inferred to make the patient feel uneasy, with use of a language model generated by machine learning, the medical information, the content information, and the state information; and
the at least one processor outputting the explanatory text.

10. A computer-readable non-transitory recording medium having recorded thereon a medical treatment assist program for causing a computer to function as a medical treatment assist apparatus, the medical treatment assist program causing the computer to carry out:
an acquiring process of acquiring medical information regarding a patient, content information which indicates content of an exchange in a medical examination between the patient and a person examining the patient, and state information regarding feelings of the patient in the medical examination;
an explanatory text generating process of generating explanatory text regarding a matter which is inferred to make the patient feel uneasy, with use of a language model generated by machine learning, the medical information, the content information, and the state information; and
an outputting process of outputting the explanatory text.
